(19) United States
(12) Patent Application Publication
(10) Pub. No.: US 2021/0030584 A1
(43) Pub. Date: Feb. 4, 2021

(54) NASOPHARYNX STENT FOR ANTI-SNORING

(71) Applicant: XIAONIU HEALTH CO., LTD., Sanhe, Hebei (CN)

(72) Inventor: Hui YAN, Sanhe, Hebei (CN)

(21) Appl. No.: 17/044,098

(22) PCT Filed: Jun. 28, 2019
(86) PCT No.: PCT/CN2019/093694

§ 371 Date: Sep. 30, 2020

(30) Foreign Application Priority Data
Jun. 29, 2018 (CN) 201821048452.7

(51) Int. Cl. A61F 5/56

(52) U.S. Cl. CPC A61F 5/56

(57) ABSTRACT

A nasopharynx stent for anti-snoring includes a nasal tube (1) configured as a soft nasal tube. The nasal tube (1) includes a middle tube body (12), a connecting tube body (13) and a rear tube body (14). The connecting tube body (13) is a curved tube. The middle tube body (12) and the rear tube body (14) are communicated with each other through the connecting tube body (13). The middle tube body (12) is provided with a plurality of second mesh-shaped through holes (120).

### CROSS-REFERENCE TO RELATED APPLICATIONS

The present application is based on and claims priority to Chinese Patent Application Serial No. 201821048452.7, filed on Jun. 29, 2018, the entire content of which is incorporated herein by reference.

### FIELD

The present application relates to a technical field of medical instruments, and more particularly, to a nasopharynx stent for anti-snoring.

### BACKGROUND

OSAHS, short for obstructive sleep apnea-hypopnea syndrome, refers to apnea and hypoventilation caused by collapse and obstruction of the upper airway during sleep, accompanied by symptoms of snoring, disturbance of sleep structure, frequent decrease in blood oxygen saturation, daytime sleepiness, etc. OSAHS has a high incidence and great harm and is closely related to multiple systemic fatal diseases throughout the body. The fact that the upper airway has a narrow structure or is susceptible to collapse is the root cause of OSAHS. OSA (obstructive sleep apnea) accounts for up to 70% of people with OSAHS. There are four types according to the obstruction site:

Type I: the stenosis is above the nasopharynx (nasopharynx and nasal cavity);

Type II: the stenosis is in the oropharynx (soft palate and tonsil/uvula level);

Type III: the stenosis is in the hypopharynx (root of tongue and epiglottis level);

Type IV: the stenosis is in each of the above sites or in more than two sites.

The type II and type III account for the highest proportion. When your body is in a relaxed state during sleep, that part of tissue will sag, compressing your airways, narrowing them and causing breathing problems (such as snoring). Stopping breathing many times a night will cause various health problems.

For OSA patients, the current treatment methods are divided into surgical treatment and non-surgical treatment. The surgical treatment is complicated, highly risky, and easy to relapse. Therefore, the recognized first choice is a non-surgical therapy of continuous airway pressure (CPAP) with a respirator, which has a wide range of treatment types. However, patients are less than 50% compliant with the respirator therapy, so that many patients are unwilling to receive treatment. The main reasons are as follows:

Uncomfortable to wear: positive pressure ventilation irritates the airways, making it difficult to exhale; claustrophobia is likely to occur when patients wear accessories on the face; fan and tube airflow noises interfere with sleep; head wearing may compress the head and cause discomfort.

Inconvenient to use: the tube is thick and long, so that it is inconvenient for users to turn over; the respirator has large volume and weight and needs to be plugged in, and with too many wires, the respirator is not portable; it is troublesome to clean and disinfect the tube and the oronasal mask, and insufficient disinfection may cause upper respiratory infection; the respirator requires humidification with purified water every time it is used.

### SUMMARY

The present disclosure seeks to solve at least one of the technical problems existing in the related art. To this end, the present disclosure proposes a nasopharynx stent for anti-snoring, which is comfortable to wear.

The nasopharynx stent for anti-snoring according to embodiments of the present disclosure includes a nasal tube configured as a soft nasal tube. The nasal tube includes a middle tube body, a connecting tube body, and a rear tube body. The connecting tube body is a curved tube. The middle tube body and the rear tube body are communicated with each other through the connecting tube body. The middle tube body is provided with a plurality of second mesh-shaped through holes.

For the nasopharynx stent according to the embodiments of the present disclosure, since the nasal tube is a soft nasal tube, and the middle tube body of the nasal tube is provided with the plurality of second mesh-shaped through holes, the second mesh-shaped through holes can allow the inhaled air to fully contact an inner wall and a mucous membrane of a nasal cavity, and help to wet the inner wall and the mucous membrane of the nasal cavity and clean the inhaled air. Therefore, the nasopharynx stent according to the embodiments of the present disclosure is comfortable to wear.

According to an embodiment of the present disclosure, the rear tube body is provided with a plurality of fourth mesh-shaped through holes. By providing the fourth mesh-shaped through holes in the rear tube body, the nasopharynx stent is more comfortable to wear.

According to some embodiments of the present disclosure, the connecting tube body is provided with a plurality of third mesh-shaped through holes. By providing the third mesh-shaped through holes in the connecting tube body, the nasopharynx stent is more comfortable to wear.

According to some embodiments of the present disclosure, a balloon is included. The balloon is a soft balloon, and the balloon is communicated with a terminal end of the rear tube body and provided with a vent hole. When the nasopharynx stent is inserted into a nasopharyngeal passage until the balloon reaches a uvula (soft palate) of the human body, the balloon may be slightly deformed due to the appropriate softness of the soft balloon and can withstand natural pressure at the uvula (soft palate), and the balloon is sufficient to support collapse of the airway at this place, such that the airway is unobstructed by means of the vent hole in the balloon, which is conducive to air inhalation and exhalation, and effectively stops snoring.

Further, the balloon is provided with a plurality of first mesh-shaped through holes. By means of the first mesh-shaped through holes in the balloon, the nasopharynx stent is more comfortable to wear.

According to some embodiments of the present disclosure, a baffle is included, and the baffle is connected to the middle tube body. By providing the baffle, the nasopharynx stent can be prevented from continuing to advance after being inserted into the nasopharyngeal passage.

Further, the middle tube body includes a first middle tube body and a second middle tube body; the connecting tube body includes a first connecting tube body and a second connecting tube body; the rear tube body includes a first rear tube body and a second rear tube body. The first middle tube body and the first rear tube body are communicated with each other through the first connecting tube body, and the second middle tube body and the second rear tube body are communicated with each other through the second connecting tube body. The first connecting tube body and the second connecting tube body are both curved tubes. The baffle includes a first baffle and a second baffle connected with the first baffle, and the first baffle and the second baffle are connected to the first middle tube body and the second middle tube body respectively.

According to some embodiments of the present disclosure, a baffle is included. The nasal tube further includes a front tube body in communication with the middle tube body. The front tube body is provided with external threads, the baffle is provided with internal threads, and the baffle is fitted over the front tube body through the fitting between the internal threads and the external threads. Since the front tube body of the nasal tube is provided with the external threads, the baffle is provided with the internal threads, and the baffle is fitted over the nasal tube through the fitting of the internal and external threads, the position of the baffle on the front tube body of the nasal tube can be adjusted by rotating the baffle, such that the effective length of the nasal tube can be adapted to different lengths of nasopharyngeal passages of different people, and the human airway is propped open and becomes unobstructed.

Further, the front tube body includes a first front tube body and a second front tube body; the middle tube body includes a first middle tube body and a second middle tube body; the connecting tube body includes a first connecting tube body and a second connecting tube body; the rear tube body includes a first rear tube body and a second rear tube body. The first front tube body and the second front tube body are both provided with external threads. The first middle tube body and the first rear tube body are communicated with each other through the first connecting tube body, and the second middle tube body and the second rear tube body are communicated with each other through the second connecting tube body. The first connecting tube body and the second connecting tube body are both curved tubes. The baffle includes a first baffle and a second baffle, and the first baffle and the second baffle are both provided with internal threads. The first baffle and the second baffle are fitted over the first front tube body and the second front tube body respectively through the fitting between the internal threads and the external threads.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and/or other aspects and advantages of embodiments of the present disclosure will become apparent and more readily appreciated from the following descriptions of embodiments made with reference to the drawings, in which:

FIG. 1 illustrates a longitudinal sectional view of a nasopharynx stent for anti-snoring according to one embodiment of the present disclosure.

FIG. 2 illustrates a schematic view of a nasopharynx stent for anti-snoring according to one embodiment of the present disclosure.

FIG. 3 illustrates a schematic view of a nasopharynx stent for anti-snoring according to another embodiment of the present disclosure.

FIG. 4 illustrates a schematic view of a nasopharynx stent for anti-snoring according to another embodiment of the present disclosure.

FIG. 5 illustrates an application scenario of a nasopharynx stent for anti-snoring according to another embodiment of the present disclosure.

FIG. 6 illustrates a schematic view of a nasopharynx stent for anti-snoring according to another embodiment of the present disclosure.

### REFERENCE NUMERALS

nasal tube 1, front tube body 11, middle tube body 12, connecting tube body 13, rear tube body 14, balloon 2, first mesh-shaped through hole 20, vent hole 21, second mesh-shaped through hole 120, fourth mesh-shaped through hole 140, baffle 14, first front tube body 111, second front tube body 112, first middle tube body 121, second middle tube body 122, first connecting tube body 131, second connecting tube body 132, first rear tube body 141, second rear tube body 142.

### DETAILED DESCRIPTION

Embodiments of the present disclosure will be described in detail below, and examples of the embodiments will be illustrated in the accompanying drawings. The same or similar reference numerals represent the same or similar elements or the elements having the same or similar functions throughout the descriptions. The embodiments described below with reference to the accompanying drawings are exemplary, are merely used to explain the present disclosure, and cannot be construed to limit the present disclosure.

In the description of the present disclosure, it should be understood that terms such as “central,” “longitudinal,” “transverse,” “length,” “width,” “thickness,” “up,” “down,” “front,” “rear,” “left,” “right,” “vertical,” “horizontal,” “top,” “bottom,” “inner,” “outer,” “clockwise,” “counterclockwise,” “axial,” “radial,” “circumferential” and the like should be construed to refer to the orientation as then described or as illustrated in the drawings under discussion. These terms are for convenience and simplification of description and do not indicate or imply that the device or element referred to must have a particular orientation, or be constructed and operated in a particular orientation, so these terms shall not be construed to limit the present disclosure. In addition, the feature defined with “first” and “second” may comprise one or more of this feature. In the description of the present disclosure, “a plurality of” means two or more than two, unless specified otherwise.

In the description of the present disclosure, it should be noted, unless specified or limited otherwise, the terms “mounted,” “connected,” “coupled” or the like are used broadly. The terms may be, for example, fixed connections, detachable connections, or integral connections; may also be mechanical or electrical connections, may also be direct connections or indirect connections via intervening structures; and may also be inner communications of two elements, which could be understood by those skilled in the art according to specific situations.

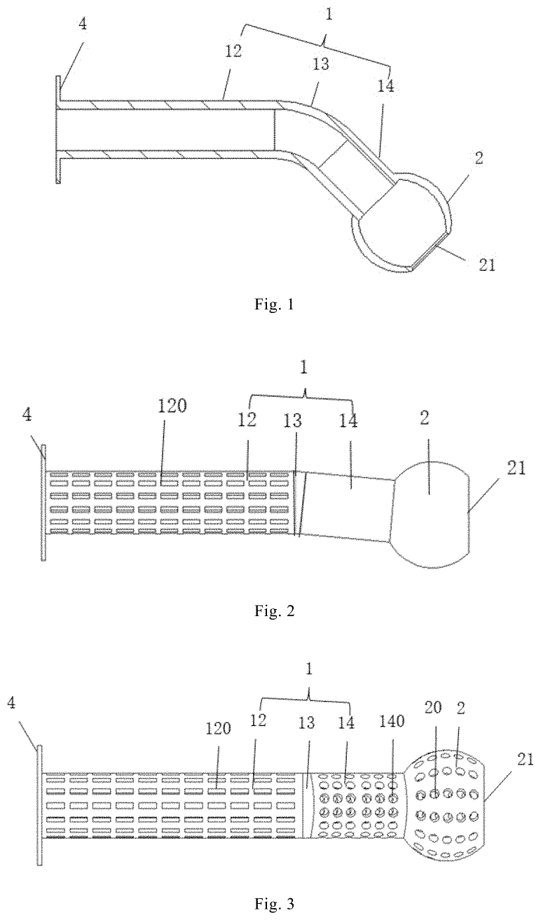

A nasopharynx stent for anti-snoring according to embodiments of the present disclosure will be described with reference to FIGS. 1-6.

As shown in FIGS. 1-4, the nasopharynx stent according to an embodiment of the present disclosure includes a nasal tube 1, and the nasal tube 1 is a soft nasal tube 1. The nasal tube 1 includes a middle tube body 12, a connecting tube body 13, and a rear tube body 14. The connecting tube body 13 is a curved tube. The middle tube body 12 and the rear tube body 14 are communicated with each other through the connecting tube body 13. The middle tube body 12 is provided with a plurality of second mesh-shaped through holes 120. The nasopharynx stent constructed as in this embodiment helps to fit the human nasopharyngeal structure, so that the nasopharynx stent of this embodiment of the present disclosure is comfortable to wear.

For the nasopharynx stent according to the embodiment of the present disclosure, since the nasal tube 1 is a soft nasal tube, and the middle tube body 12 of the nasal tube 1 is provided with the plurality of second mesh-shaped through holes 120, the second mesh-shaped through holes 120 can allow the inhaled air to fully contact an inner wall and a mucous membrane of a nasal cavity, and help to wet the inner wall and the mucous membrane of the nasal cavity and clean the inhaled air. Therefore, the nasopharynx stent of the embodiment of the present disclosure is comfortable to wear.

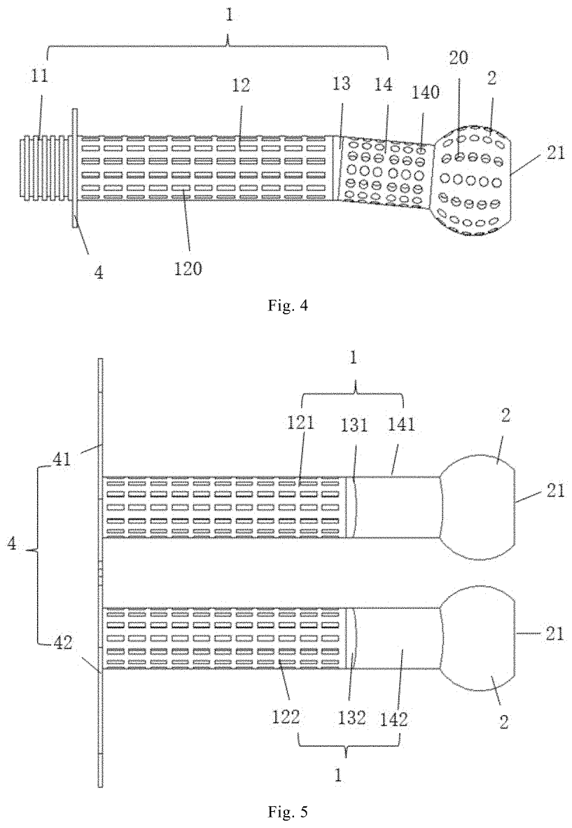

As shown in FIG. 3 and FIG. 4, according to some embodiments of the present disclosure, the rear tube body 14 is provided with a plurality of fourth mesh-shaped through holes 140. By providing the fourth mesh-shaped through holes 140 in the rear tube body 14, the nasopharynx stent is more comfortable to wear.

According to some embodiments of the present disclosure, the connecting tube body 13 is provided with a plurality of third mesh-shaped through holes (not shown in the drawings). By providing the third mesh-shaped through holes in the connecting tube body 13, the nasopharynx stent is more comfortable to wear.

As shown in FIGS. 1-4, according to some embodiments of the present disclosure, a balloon 2 is included, and the balloon 2 is a soft balloon. The balloon 2 is in communication with a terminal end of the rear tube body 14, and the balloon 2 is provided with a vent hole 21. When the nasopharynx stent is inserted into a nasopharyngeal passage until the balloon 2 reaches a uvula (soft palate) of the human body, the balloon may be slightly deformed due to the appropriate softness of the soft balloon 2 and can withstand natural pressure at the uvula (soft palate), and the balloon 2 is sufficient to support collapse of the airway at this place, such that the airway is unobstructed by means of the vent hole 21 in the balloon 2, which is conducive to air inhalation and exhalation, and effectively stops snoring.

As shown in FIG. 3 and FIG. 4, according to further embodiments of the present disclosure, the balloon 2 is provided with a plurality of first mesh-shaped through holes 20. By means of the first mesh-shaped through holes 20 in the balloon, the nasopharynx stent is more comfortable to wear.

As shown in FIGS. 1-3, according to some embodiments of the present disclosure, the nasopharynx stent includes a baffle 4, and the baffle 4 is connected to the middle tube body 12. By providing the baffle 4, the nasopharynx stent can be prevented from continuing to advance after being inserted into the nasopharyngeal passage.

As shown in FIG. 5, according to an embodiment of the present disclosure, the middle tube body 12 includes a first middle tube body 121 and a second middle tube body 122; the connecting tube body 13 includes a first connecting tube body 131 and a second connecting tube body 132; the rear tube body 14 includes a first rear tube body 141 and a second rear tube body 142. The first middle tube body 121 and the first rear tube body 141 are communicated with each other through the first connecting tube body 131, and the second middle tube body 122 and the second rear tube body 142 are communicated with each other through the second connecting tube body 132. The first connecting tube body 131 and the second connecting tube body 132 are both curved tubes. The baffle 4 includes a first baffle 41 and a second baffle 42 connected with the first baffle 41. The first baffle 41 and the second baffle 42 are connected to the first middle tube body 121 and the second middle tube body 122, respectively. Respective terminal ends of the first rear tube body 14 and the second rear tube body 142 are each connected with one balloon 2, and the balloon 2 is provided with the vent hole 21 and the first mesh-shaped through holes 20.

As shown in FIG. 4, according to an embodiment of the present disclosure, the nasopharynx stent further includes a baffle 4. The nasal tube 1 further includes a front tube body 11, and the front tube body 11 is in communication with the middle tube body 12. The front tube body 11 is provided with external threads, and the baffle 4 is provided with internal threads. The baffle 4 is fitted over the front tube body 11 through the fitting between the internal threads and the external threads. Since the front tube body 11 of the nasal tube 1 is provided with the external threads, the baffle 4 is provided with the internal threads, and the baffle 4 is fitted over the nasal tube 1 through the fitting of the internal and external threads, the position of the baffle 4 on the front tube body 11 of the nasal tube 1 can be adjusted by rotating the baffle 4, such that the effective length of the nasal tube 1 can be adapted to different lengths of nasopharyngeal passages of different people, and the human airway is propped open and becomes unobstructed.

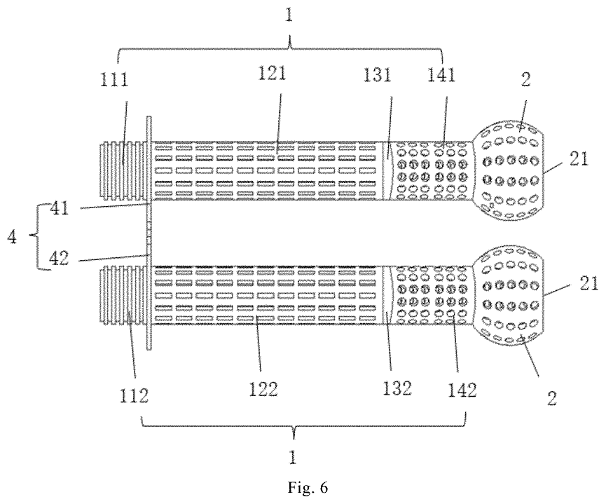

As shown in FIG. 6, according to an embodiment of the present disclosure, the front tube body 11 includes a first front tube body 111 and a second front tube body 112; the middle tube body 12 includes a first middle tube body 121 and a second middle tube body 122; the connecting tube body 13 includes a first connecting tube body 131 and a second connecting tube body 132; the rear tube body 14 includes a first rear tube body 141 and a second rear tube body 142. The first front tube body 111 and the second front tube body 112 are both provided with external threads. The first middle tube body 121 and the first rear tube body 141 are communicated with each other through the first connecting tube body 131, and the second middle tube body 122 and the second rear tube body 142 are communicated with each other through the second connecting tube body 132. The first connecting tube body 131 and the second connecting tube body 132 are both curved tubes. The baffle 4 includes a first baffle 41 and a second baffle 42, and the first baffle 41 and the second baffle 42 are both provided with internal threads. The first baffle 41 and the second baffle 42 are fitted over the first front tube body 111 and the second front tube body 112 respectively through the fitting of the internal and external threads. Respective terminal ends of the first rear tube body 14 and the second rear tube body 142 are each connected with one balloon 2, and the balloon 2 is provided with the vent hole 21 and the first mesh-shaped through holes 20.

Reference throughout this specification to “an embodiment,” “some embodiments,” “an exemplary embodiment,” “an example,” “a specific example,” or “some examples,” means that a particular feature, structure, material, or characteristic described in connection with the embodiment or example is included in at least one embodiment or example of the present disclosure. Thus, the appearances of the phrases in various places throughout this specification are not necessarily referring to the same embodiment or example of the present disclosure. Moreover, the particular features, structures, materials, or characteristics described may be combined in any suitable manner in one or more embodiments or examples.

Although embodiments of the present disclosure have been illustrated and described above, it could be understood by those skilled in the art that changes, modifications, alternatives, and variations can be made in the embodiments without departing from the scope of the present disclosure.

1. A nasopharynx stent for anti-snoring, comprising:
a nasal tube configured as a soft nasal tube, and comprising a middle tube body, a connecting tube body, and a rear tube body, the connecting tube body being a curved tube, the middle tube body and the rear tube body being communicated with each other through the connecting tube body, and the middle tube body being provided with a plurality of second mesh-shaped through holes.

2. The nasopharynx stent according to claim 1, wherein the rear tube body is provided with a plurality of fourth mesh-shaped through holes.

3. The nasopharynx stent according to claim 1, wherein the connecting tube body is provided with a plurality of third mesh-shaped through holes.

4. The nasopharynx stent according to claim 1, further comprising a balloon configured as a soft balloon, the balloon being communicated with a terminal end of the rear tube body and provided with a vent hole.

5. The nasopharynx stent according to claim 4, wherein the balloon is provided with a plurality of first mesh-shaped through holes.

6. The nasopharynx stent according to claim 1, further comprising a baffle connected to the middle tube body.

7. The nasopharynx stent according to claim 6, wherein the middle tube body comprises a first middle tube body and a second middle tube body; the connecting tube body comprises a first connecting tube body and a second connecting tube body; the rear tube body comprises a first rear tube body and a second rear tube body, wherein the first middle tube body and the first rear tube body are communicated with each other through the first connecting tube body, and the second middle tube body and the second rear tube body are communicated with each other through the second connecting tube body; the first connecting tube body and the second connecting tube body are both curved tubes; the baffle comprises a first baffle and a second baffle connected with the first baffle, and the first baffle and the second baffle are connected to the first middle tube body and the second middle tube body respectively.

8. The nasopharynx stent according to claim 1, further comprising a baffle, wherein the nasal tube further comprises a front tube body in communication with the middle tube body; the front tube body is provided with external threads, the baffle is provided with internal threads, and the baffle is fitted over the front tube body through the fitting between the internal threads and the external threads.

9. The nasopharynx stent according to claim 8, wherein the front tube body comprises a first front tube body and a second front tube body; the middle tube body comprises a first middle tube body and a second middle tube body; the connecting tube body comprises a first connecting tube body and a second connecting tube body; the rear tube body comprises a first rear tube body and a second rear tube body, wherein the first front tube body and the second front tube body are both provided with external threads; the first middle tube body and the first rear tube body are communicated with each other through the first connecting tube body, and the second middle tube body and the second rear tube body are communicated with each other through the second connecting tube body; the first connecting tube body and the second connecting tube body are both curved tubes; the baffle comprises a first baffle and a second baffle, and the first baffle and the second baffle are both provided with internal threads; the first baffle and the second baffle are fitted over the first front tube body and the second front tube body respectively through the fitting between the internal threads and the external threads.

10. The nasopharynx stent according to claim 7, wherein respective terminal ends of the first rear tube body and the second rear tube body are each connected with a balloon, and the balloon is provided with a vent hole and first mesh-shaped through holes.

11. The nasopharynx stent according to claim 9, wherein respective terminal ends of the first rear tube body and the second rear tube body are each connected with a balloon, and the balloon is provided with a vent hole and first mesh-shaped through holes.

12. The nasopharynx stent according to claim 4, wherein the soft balloon has an appropriate degree of softness to withstand a natural pressure at a uvula and support collapse of an airway.

13. The nasopharynx stent according to claim 8, wherein a position of the baffle on the front tube body of the nasal tube can be adjusted by rotating the baffle, and an effective length of the nasal tube can be adjusted.
